(19) United States
(12) Patent Application Publication
(10) Pub. No.: US 2026/0157974 A1
(43) Pub. Date: Jun. 11, 2026

(54) DRUG-RELEASING NANOPARTICLE-ENHANCED HYDROGEL, PREPARATION METHOD THEREFOR, AND USE THEREOF

(71) Applicant: THE HONG KONG UNIVERSITY OF SCIENCE AND TECHNOLOGY, Kowloon (CN)

(72) Inventors: Ziyi ZHANG, Kowloon (CN); Weijia WEN, Kowloon (CN)

(21) Appl. No.: 19/410,725

(22) Filed: Dec. 5, 2025

(30) Foreign Application Priority Data
Dec. 6, 2024 (CN) 202411794060.5

(51) Int. Cl. A61K 9/51; A61K 9/06; A61K 31/167; A61K 31/445; A61K 31/616; A61P 17/02

(52) U.S. Cl. CPC A61K 9/5146; A61K 9/06; A61K 31/167; A61K 31/445; A61K 31/616; A61P 17/02

(57) ABSTRACT

The present disclosure discloses a drug-releasing nanoparticle-enhanced hydrogel, a preparation method therefor and use thereof. The drug-releasing hydrogel comprises: a drug nanoparticle, comprising an active pharmaceutical ingredient encapsulated by a phospholipid-acrylate polymer; and a porous hydrogel formed by crosslinking an acrylated biodegradable polymer with a pore-forming agent; wherein the porous hydrogel is loaded with the drug nanoparticle. The drug-releasing nanoparticle-enhanced hydrogel can achieve more than 90% of drug release, which can be adjusted within 4 to 72 hours, significantly improving a wound healing rate and skin regeneration. The hydrogel is biocompatible, is capable of promoting re-epithelialization, angiogenesis, and collagen regeneration, and results in minimal scarring.

### TECHNICAL FIELD

The present disclosure relates to the technical field of medicine, and in particular to a drug-releasing nanoparticle-enhanced hydrogel, a preparation method therefor, and use thereof.

### BACKGROUND

Diabetic wounds pose significant challenges due to impaired angiogenesis and dysregulated inflammation, leading to chronic wounds. However, it remains challenging for traditional wound dressings to meet the complex healing process. Currently, standard clinical wound dressings (e.g., gauze and films) are not adequately adapted to the diabetic wound environment because they neither continuously release active substances nor accelerate the wound healing process, and often encounter the risk of adhesion and damage to diabetic wounds. Hydrogels contain a large amount of water, providing a moist environment for patients and can be used as wound dressings. The hydrogels have structural similarities to soft tissues, and can incorporate therapeutic ingredients that promote diabetic wound healing. Composite hydrogels, if directly mixed with particle suspensions, will have enhanced functionality, as demonstrated by different approaches. However, these hydrogel therapeutic platforms have limitations, including uncontrolled release, complex reaction conditions for pre-treatment, and changes in hydrogel hydrophilicity.

Drug-loaded biomaterials have emerged as a promising platform for diabetic wound healing. Aspirin, as one of the most potent nonsteroidal anti-inflammatory drugs (NSAIDs), inhibits the cyclooxygenase-2 (COX-2) enzyme. COX-2 is a known mediator of prostaglandin E2 (PGE2) secretion, which is involved in promoting inflammatory conditions. The alleviation of the early inflammatory phase through COX-2 and PGE2 inhibition significantly impacts the diabetic wound healing process. In parallel, local anesthetics (LAs) suppress the production of inflammatory mediators including nitric oxide (NO), PGE2, tumor necrosis factor (TNF)-α, interleukin (IL)-6 and IL-1β. The clinical administration of LAs to diabetic foot ulcers has demonstrated dual benefits: significantly reducing wound pain during and after dressing changes, while promoting wound healing through the inhibition of the inflammatory PGE2 pathway via TNF-α suppression. However, both therapeutic approaches face limitations. Conventional LA administration typically only provides 2-4 hours of drug efficacy, and while the combination of lidocaine and ropivacaine shows improved postoperative analgesia compared to single compounds, the acidosis present in inflamed tissues can diminish LA efficacy by reducing their ability to penetrate cell membranes. A transdermal absorbable adhesive patch has been developed, which comprises a basic anti-inflammatory analgesic and a local anesthetic as an absorption enhancer. However, there is still a lack of an effective drug delivery method to achieve multi-drug compatibility and a long-term therapeutic effect. Therefore, it is crucial to improve the quality of life of diabetic patients by developing composite materials with comprehensive functions to accelerate wound healing.

### SUMMARY

The present disclosure aims to solve at least one of the aforementioned technical problems existed in the prior art. To this end, an objective of the present disclosure is to provide a drug-releasing nanoparticle-enhanced hydrogel, a preparation method therefor, and use thereof.

In order to achieve the aforementioned objective, the technical solution adopted by the present disclosure is as follows.

In a first aspect of the present disclosure, a drug-releasing hydrogel is provided, comprising:
a drug nanoparticle, comprising an active pharmaceutical ingredient encapsulated by a phospholipid-acrylate polymer;
a porous hydrogel formed by crosslinking an acrylated biodegradable polymer with a pore-forming agent;
wherein the porous hydrogel is loaded with the drug nanoparticle.

In the present disclosure, the phospholipid-acrylate polymer exhibits excellent biocompatibility, encapsulates the active pharmaceutical ingredient to form a regularly shaped spherical nanoparticle (DNP), and provides uniform assembly and outer layer distribution of acrylate for the drug nanoparticle, thereby increasing the zeta potential value of the drug nanoparticle, and thus improving the stability of distribution of the drug nanoparticle in water. On the other hand, the introduction of the pore-forming agent into the acrylated biodegradable polymer can create pores into the hydrogel, which enhances the accessibility of loading of the drug nanoparticle. The drug nanoparticle comprises an acrylate group that can crosslink with the acrylated biodegradable polymer in the porous hydrogel to improve the crosslinking density of the drug-releasing hydrogel, and thus enhance the pore structure of the hydrogel and promote the stable loading of the drug nanoparticle in the hydrogel, thereby achieving long-lasting release of the active pharmaceutical ingredient. Additionally, the aforementioned crosslinking can limit the swelling performance of the hydrogel. The low swelling ratio can effectively absorb a wound exudate and maintain the mechanical performance that a dressing should have.

In some embodiments of the present disclosure, in the drug-releasing hydrogel, a mass ratio of the drug nanoparticle to the porous hydrogel is 1-30: 70-99, e.g., 5-28: 72-95, 10-25: 75-90, 12-25: 75-88, 15-25: 75-85, and 18-23: 72-87.

In some embodiments of the present disclosure, in the drug nanoparticle, a mass ratio of the phospholipid-acrylate polymer to the active pharmaceutical ingredient is 1:0.1-10, e.g., 1:0.3-8, 1:0.5-7, 1:0.8-5, 1:1-4, etc.

In some embodiments of the present disclosure, in the porous hydrogel, a mass of the pore-forming agent accounts for 20-90%, e.g., 30%, 35%, 40%, 45%, 50%, 55%, 60%, 65%, 70%, 75%, 80%, 85%, 90%, etc., of a total weight of the porous hydrogel. In the present disclosure, a porosity in the porous hydrogel can be regulated by adjusting the mass ratio of the pore-forming agent, thereby regulating the drug loading amount and mechanical strength, and thus regulating the drug release behavior of the drug-releasing hydrogel according to actual needs.

In some embodiments of the present disclosure, the phospholipid-acrylate polymer comprises a polymer formed by phospholipid, polyethylene glycol and acrylic acid, wherein the acrylic acid chemically reacts with a hydroxyl group at an end of the polyethylene glycol chain to form an ester bond. This structure enables the phospholipid-acrylate polymer to possess both water solubility of the PEG chain and reactivity of the acrylic acid. It not only can exist stably in an aqueous solution, but also can effectively bind with a hydrophobic ingredient in an organism, showing an excellent balance between hydrophilicity and hydrophobicity.

In some embodiments of the present disclosure, the phospholipid comprises at least one of 1,2-distearoyl-sn-glycero-3-phosphoethanolamine (DSPE); dipalmitoyl-phosphatidylethanolamine (DPPE); distearoylphosphocholine (DSPC); dimyristoyl-phosphatidylethanolamine (DMPE); 1,2-dioleoyl-sn-glycero-3-phosphoethanolamine (DOPE); 1,2-dioleoyl-sn-glycero-3-phosphocholine (DOPC); PEGylated phospholipids selected from the group consisting of DSPE-PEG, DPPE-PEG, DMPE-PEG, and DOPE-PEG; polymer-phospholipid conjugates selected from the group consisting of phospholipid-chitosan conjugate, phospholipid-hyaluronic acid conjugate, phospholipid-dextran conjugate, and phospholipid-poly(lactide-co-glycolide) conjugate.

In some embodiments of the present disclosure, PEG of the PEGylated phospholipids has a molecular weight of 1,000 Da to 10,000 Da.

In some embodiments of the present disclosure, a molecular weight of the polyethylene glycol is 300 Da to 25,000 Da, and preferably 500 Da to 5,000 Da.

In some embodiments of the present disclosure, a molecular weight of the polyethylene glycol is 1,000 Da to 25,000 Da, and preferably 1,000 Da to 5,000 Da.

In some embodiments of the present disclosure, the acrylated biodegradable polymer comprises at least one of polyethylene glycol (PEG) acrylate (PEGA), PEG methacrylate (PEGMA), PEG diacrylate (PEGDA), disulfide-containing PEGDA (PEGSSDA), PEG dimethacrylate (PEGDMA), poly(2-hydroxyethyl methacrylate) (pHEMA), poly(acrylic acid) (PAA), polyacrylate, poly(methacrylic acid) (PMA), or polymethacrylate.

In some embodiments of the present disclosure, a number average molecular weight of the polyethylene glycol diacrylate is 500-8,000, for example, 500, 525, 550, 575, 600, 650, 700, 750, 800, 850, 900, 950, 1,000, 1,050, 1,100, 1,150, 1,200, 1,500, 2,000, 2,500, 3,000, 3,500, 4,000, 4,500, 5,000, 6,000, 7,000, 8,000, etc.

In some embodiments of the present disclosure, the pore-forming agent comprises at least one of polyethylene glycol (PEG), chitosan, agarose, dextran, hyaluronic acid, poly(methyl methacrylate) (PMMA), cellulose and a derivative thereof, gelatin and a derivative thereof, or acrylamide and a derivative thereof.

In some embodiments of the present disclosure, the cellulose and a derivative thereof comprise at least one of methyl cellulose, ethyl cellulose, ethyl methyl cellulose, hydroxypropyl methyl cellulose, hydroxypropyl cellulose, or hydroxyethyl cellulose.

In some embodiments of the present disclosure, the active pharmaceutical ingredient comprises an anti-inflammatory drug, including nonsteroidal anti-inflammatory drugs (NSAIDs), such as diclofenac, etodolac, fenoprofen, flurbiprofen, ibuprofen, indomethacin, ketorolac, meclofenamic acid, mefenamic acid, nabumetone, naproxen, oxaprozin, piroxicam, sulindac, tolmetin, celecoxib, meloxicam, rofecoxib, valdecoxib, aspirin; and steroidal anti-inflammatory drugs, including cortisone, prednisone, and dexamethasone.

In some embodiments of the present disclosure, the active pharmaceutical ingredient comprises an analgesic, including but not limited to acetaminophen, anileridine, acetylsalicylic acid, buprenorphine, butorphanol, fentanyl, fentanyl citrate, codeine, rofecoxib, hydrocodone, hydromorphone, hydromorphone hydrochloride, levorphanol, alfentanil hydrochloride, meperidine, meperidine hydrochloride, methadone, morphine, nalbuphine, opium, levomethadyl, sodium hyaluronate, sufentanil citrate, capsaicin, tramadol, leflunomide, oxycodone, oxymorphone, celecoxib, pentazocine, propoxyphene, benzocaine, lidocaine, dezocine, clonidine, butalbital, phenobarbital, and tetracaine.

In some embodiments of the present disclosure, the active pharmaceutical ingredient comprises a local anesthetic, including amylocaine, ambucaine, articaine, benzocaine, benzonatate, bupivacaine, tetracaine, butanilicaine, chloroprocaine, cinchocaine, cyclomethycaine, dibucaine, diperodon, dimethisoquin, dimethocaine, eucaine, etidocaine, hexylcaine, fomocaine, fotocaine, hydroxyprocaine, isobucaine, levobupivacaine, iodocaine, mepivacaine, meprylcaine, metabutoxycaine, nitracaine, orthocaine, oxetacaine, oxybuprocaine, parethoxycaine, phenacaine, piperocaine, piridocaine, pramocaine, prilocaine, primacaine, procaine, procainamide, proparacaine, propoxycaine, pyrrocaine, quinisocaine, ropivacaine, trimecaine, tetracaine, tolycaine, and tropacocaine.

In some embodiments of the present disclosure, the active pharmaceutical ingredient comprises at least one of an anti-inflammatory drug, an analgesic, or a local anesthetic. To promote wound healing, multiple active pharmaceutical ingredients, e.g., anti-inflammatory drugs, analgesics and local anesthetics, can be used together simultaneously. For example, aspirin, lidocaine and ropivacaine are used simultaneously.

In some embodiments of the present disclosure, the active pharmaceutical ingredient may further comprise a cytokine, a growth factor, any wound healing agonist (or effective wound healing agent), including but not limited to a small molecule agonist, a peptide agonist, a chemical agonist or a mixture thereof. In some embodiments of the present disclosure, the growth factor can be, for example, a platelet-derived growth factor, a vascular endothelial growth factor, a fibroblast growth factor, an epidermal growth factor, TGF-β, and a mixture thereof.

In some embodiments of the present disclosure, the drug nanoparticle is spherical or quasi-spherical, and has a dimension of 50-200 nm, preferably 80-200 nm, e.g., 100-180 nm.

In some embodiments of the present disclosure, a zeta potential value of the drug nanoparticle is −35 mV to −5 mV, preferably −35 mV to −20 mV, e.g., −30 mV to −22 mV.

In some embodiments of the present disclosure, the drug-releasing hydrogel can be of any shape, e.g., a bulk structure, a thin film, or microhydrogel, etc. A microhydrogel is a crosslinked network particle with a dimension between submicron and micron. It is characterized by a large specific surface area, strong mechanical performance for oscillation in fluid, a slow drug release rate and a prolonged drug release duration.

In some embodiments of the present disclosure, a diameter of the pores in the porous hydrogel is 1-100 μm, e.g., 50-100 μm, 1-35 μm, 5-30 μm, 10-30 μm, 1-10 μm, etc.

In a second aspect of the present disclosure, a method for preparing the drug-releasing hydrogel is provided, comprising the following steps:

adding a pore-forming agent, a drug nanoparticle solution and an initiator into an acrylated biodegradable polymer solution, and performing light curing to prepare the drug-releasing hydrogel.

In some embodiments of the present disclosure, the drug nanoparticle solution is prepared by mixing an organic solution of a phospholipid-acrylate polymer with an organic solution of an active pharmaceutical ingredient, then dispersing a mixture of the two solutions into water, and sonicating the mixture to obtain the drug nanoparticle solution.

In some embodiments of the present disclosure, a volume ratio of an organic solvent to water is 1:5-100.

In some embodiments of the present disclosure, a mass concentration of the phospholipid-acrylate polymer in the organic solution of the phospholipid-acrylate polymer is 0.01 wt %-1.0 wt %, e.g., 0.03 wt %-0.8 wt %, 0.05 wt %-0.6 wt %, 0.1 wt %-0.5 wt %, etc.

In some embodiments of the present disclosure, a mass concentration of the active pharmaceutical ingredient in the organic solution of the active pharmaceutical ingredient is 0.01 wt %-1.0 wt %, e.g., 0.03 wt %-0.8 wt %, 0.05 wt %-0.6 wt %, 0.1 wt %-0.5 wt %, etc.

In some embodiments of the present disclosure, the organic solvent of the drug nanoparticle solution is removed, aggregates are removed by filtration, and the excess phospholipid-acrylate polymer is removed by dialysis to obtain a uniformly dispersed drug nanoparticle solution.

In some embodiments of the present disclosure, a mass concentration of the acrylated biodegradable polymer in the acrylated biodegradable polymer solution is 1 wt %-50 wt %, e.g., 5 wt %-45 wt %, 10 wt %-40 wt %, 15 wt %-30 wt %, 20 wt %-25 wt %, etc.

In some embodiments of the present disclosure, a mass concentration of the drug nanoparticle in the drug nanoparticle solution is 0.01 wt %-1.0 wt %, e.g., 0.03 wt %-0.8 wt %, 0.05 wt %-0.6 wt %, 0.1 wt %-0.5 wt %, etc.

In some embodiments of the present disclosure, the organic solvent comprises at least one of dimethyl sulfoxide (DMSO), acetone, tetrahydrofuran (THF), ethanol, methanol, N,N-dimethylformamide (DMF), N-methyl-2-pyrrolidone (NMP), dimethylacetamide (DMAc), 1,4-dioxane, chloroform, dichloromethane (DCM), acetonitrile, isopropanol, ethyl acetate, or mixtures thereof.

In some embodiments of the present disclosure, the initiator comprises at least one of 2-hydroxy-2-methyl-1-phenyl-1-propanone (Darocur 1173), (2,4,6-trimethylbenzoyl)diphenylphosphine oxide (TPO), 1-hydroxycyclohexylphenyl ketone (184), 2,2-dimethoxy-phenylacetophenone (BDK), benzophenone (BP), 2-isopropylthiothianthone (2,4 isomer mixture) (ITX), 2-methyl-1-(4-methylthiophenyl)-2-morpholino-1-propanone (907), 2-benzyl-2-dimethylamino-1-(4-morpholinophenyl)butanone (369), phenylbis(2,4,6-trimethylbenzoyl)phosphine oxide (819), a benzoyl formate mixture (754), 1,1′-(methylenebis(4,1-phenylene))bis(2-hydroxy-2-methylpropan-1-one) (Irgacure 127), 2-hydroxy-4′-(2-hydroxyethoxy)-2-methylpropiophenone (Irgacure 2959), lithium phenyl(2,4,6-trimethylbenzoyl)phosphinate (LAP), camphorquinone (CQ), ammonium persulfate (APS), 2,2′-azobis(2-methylpropionamidine) dihydrochloride (V-50), riboflavin, or a combination of ammonium persulfate and N,N,N′,N′-tetramethylethylenediamine (APS/TEMED).

In some embodiments of the present disclosure, a mass ratio of the acrylated biodegradable polymer to the initiator is 5-50:1, e.g., 10-40:1, 20-30:1, etc.

In some embodiments of the present disclosure, the sonicating is conducted at a frequency of 20 KHz-60 KHz (e.g., 30 KHz, 40 KHz, 50 KHz) and an ultrasound power of 80-130 W (e.g., 90 W, 100 W, 110 W, 120 W) for a duration of 1 min-10 min, e.g., 3 min, 5 min, 8 min, etc.

In some embodiments of the present disclosure, the light curing is performed by crosslinking under ultraviolet light.

In some embodiments of the present disclosure, illumination is conducted with the ultraviolet light at a light intensity of 80-150 mJ/cm² (e.g., 90 mJ/cm², 100 mJ/cm², 110 mJ/cm², 120 mJ/cm², 130 mJ/cm², 140 mJ/cm²) for 30 s-5 min.

In some embodiments of the present disclosure, a wavelength of the illumination is 250-380 nm, e.g., 280-350 nm, 300-330 nm, etc.

In a third aspect of the present disclosure, use of the drug-releasing hydrogel in preparation of a drug for wound healing is provided.

In some embodiments of the present disclosure, the wound comprises at least one of a burn, a trauma, a wound left by surgical intervention (e.g., a dental operation, other surgical operations, etc.), a chronic wound, e.g., an ulcerative wound, or a diabetic wound, such as a diabetic ulcer wound.

In a fourth aspect of the present disclosure, a device for wound is provided, comprising the drug-releasing hydrogel, and optionally a substrate material.

In some embodiments of the present disclosure, the drug-releasing hydrogel can be provided in a desired dimension and shape by cutting a substrate material into a desired dimension and shape and peeling a drug-releasing hydrogel sheet off from the substrate. The drug-releasing hydrogel can then be applied to a biological surface (e.g., a wound) or a medical surface (e.g., a surface of a medical device (e.g., wound coverings). In some embodiments, the drug-releasing hydrogel is used for modifying wound dressings or biological wound dressings that are compatible with functionalization achieved by the addition of a matrix material. Examples of commercially available wound dressings that can be modified by the addition of the drug-releasing hydrogel include, but are not limited to, Biobrane™, gauze, tapes, bandages such as Band-Aids®, and other commercially available wound dressings including, but are not limited to, COMPEEL®, DUODERM™, TAGADERM™, and OPSITE®. In some embodiments, the present disclosure provides a method for transferring the drug-releasing hydrogel to a desired surface (e.g., a soft surface). Such a soft surface includes, but is not limited to, skin, a wound bed, a tissue, an artificial tissue, including an artificial skin tissue such as an organ-cultured skin tissue, Apligraf®, Dermagraft®, Oasis®, Transcyte®, Cryoskin®, and Myskin®, an artificial tissue matrix, a gel containing a biomolecule, a wound dressing, and a biological wound dressing. In some embodiments, the desired surface is contacted with the drug-releasing hydrogel, e.g., a drug-releasing hydrogel supported on a carrier, and pressure is applied to effect transfer of the drug-releasing hydrogel from the carrier to the desired surface. In some embodiments, the pressure is from about 10 kPa to about 500 kPa. In some embodiments, the transfer is performed in the substantial or complete absence of a solution. This dry transfer process does not involve exposing the biological component of the desired surface to an aqueous solution containing a substance that may affect the activity of the biological component. In some embodiments, the transfer is performed via a gas phase. In some embodiments, the transfer is performed in an environment where the humidity is less than 100% saturation. In some embodiments, the transfer is performed in the absence of liquid water.

In some embodiments, the drug-releasing hydrogel is used for modifying an adhesive bandage comprising an adhesive portion (e.g., an adhesive strip) and an absorbent material. Preferably, the adhesive bandage is treated or coated with a material (i.e., a non-adhesive material) to prevent adhesion to the adhesive portion. A surface of the wound or an absorbent pad to be contacted with the wound comprises a layer of non-adhesive material, e.g., Teflon®. In some embodiments, a supporting material is an absorbent pad (e.g., a gauze pad or polymer foam), which is preferably treated or coated with a material (i.e., a non-adhesive material) to prevent adherence to the wound or comprises a non-adhesive layer. The adhesive material on the surface of the absorbent pad that contacts the wound, is e.g., Teflon® or other suitable material. In some embodiments, the non-adhesive material or layer is breathable. In some embodiments, the wound dressing comprises a gel-forming agent, for example a hydrocolloid, e.g., sodium carboxymethylcellulose. In some embodiments, the absorbent pad or the gel-forming agent is fixed to a waterproof and/or breathable material. Examples include, but are not limited to, a semipermeable polyurethane membrane. The waterproof and/or breathable material may also comprise an adhesive material for fixing a bandage to the skin of a subject. The waterproof and/or breathable material preferably forms an outer surface of the adhesive bandage or pad, i.e., the surface opposite to the surface comprising the matrix that contacts the wound.

Examples of such adhesive bandages and absorbent pads include, but are not limited to, adhesive bandages and pads from the Band-Aid® series of wound dressings; adhesive bandages and pads from the Nexcare® series of wound dressings; adhesive bandages and non-adhesive bandages; adhesive pads from the Kendall Curity Tefla® series of wound dressings; adhesive bandages and pads from the Tegaderm® series of wound dressings; adhesive bandages and pads from the Steri-Strip® series of wound dressings; wound dressings, adhesive bandages and pads from the COMFEEL® series; wound dressings, adhesive bandages and pads from the Duoderm® series; wound dressings, adhesive bandages and pads from the TEGADERM™ series; wound dressings, adhesive bandages and pads from the OPSITE® series; adhesive bandages and pads from the Allevyn™ series of wound dressings; adhesive bandages and pads from the Duoderm® series of wound dressings; and adhesive bandages and pads from the Xeroform® series of wound dressings.

In some embodiments, the device of the present disclosure is used for modifying a medical device, e.g., a surgical mesh. Examples of commercially available surgical meshes that can be modified by the addition of a matrix as described hereafter include, but are not limited to, polypropylene, polyester, polytetrafluoroethylene meshes, or absorbable biological meshes or biological meshes (biomeshes), including, but are not limited to, ULTRAPRO™ mesh fabrics, PROCEED™ mesh fabrics, PROLENE™ polypropylene mesh fabrics, Ethicon Physiomesh™ MERSILENE™ polyester mesh fabrics, PARIETEX™ mesh fabrics, DOLPHIN™ polypropylene mesh fabrics, GORE INFINIT™ mesh fabrics, PERFIX™, KUGEL™, 3DMAX™, BARD™ VISILEX™, XENMATRIX™, ALLOMAX™, SURGISIS BIODESIGN™, and TIGR MATRIX™.

The beneficial effects of the present disclosure are as follows.

The drug-releasing nanoparticle-enhanced hydrogel of the present disclosure has a well-defined pore structure, which is intended to achieve sustainable drug release and effective wound healing, especially for diabetic patients. The hydrogel may comprise surface-modified anti-inflammatory and local anesthetic drug nanoparticles that are crosslinked with a gel precursor to enhance structural integrity and provide sustained drug release. This drug delivery system can achieve more than 90% of drug release, which can be adjusted within 4 to 72 hours, significantly improving a wound healing rate and skin regeneration. The hydrogel is biocompatible, is capable of promoting re-epithelialization, angiogenesis, and collagen regeneration, and results in minimal scarring. The present disclosure solves the technical problem of providing a moist environment for wound healing while ensuring controlled drug release and structural stability, making it very effective for chronic wound management.

The nanoparticle-enhanced hydrogel described in the present disclosure has the following advantages over the existing hydrogel platforms.
1. It provides efficient drug nanoparticle preparation and simple drug compatibility.
2. Structural integrity is enhanced due to crosslinking of surface-modified drug nanoparticles with hydrogel matrix.
3. It provides controlled and sustained drug release, which can be adjusted within 4 to 72 hours.
4. It improves the wound healing rate and the skin regeneration rate, the healing rate being 17 times that of untreated wounds.
5. It provides effective anti-inflammatory and analgesic properties, promoting re-epithelialization, angiogenesis and collagen regeneration.

### BRIEF DESCRIPTION OF DRAWINGS

FIG. 1 is an illustration of an anti-inflammatory mechanism of a sustainable treatment platform of the technical solution of the present disclosure.

FIG. 2 shows a scheme for preparation of DNPs and a nanodrug-hydrogel composite.

FIGS. 3a-3f show SEM and DLS measurements of drug nanoparticles; FIGS. 3a-3c show typical SEM images and DLS size distribution of F-127-encapsulated DNPs (aspirin (FIG. 3a), lidocaine (FIG. 3b), and ropivacaine (FIG. 3c)); and FIGS. 3d-3f show typical SEM images and DLS size distribution of DSPE-PEG-AC-encapsulated DNPs-AC (aspirin (FIG. 3d), lidocaine (FIG. 3e), and ropivacaine (FIG. 3f)).

FIGS. 4a-4b show Zeta potential of DNPs without acrylate and with acrylate in water.

FIGS. 5a-5d show the morphology and drug release characteristics of drug-loaded hydrogels; FIG. 5a shows a typical SEM image of a gel loaded with drug nanoparticles that are not surface-modified with acrylate (Gel-DNPs); FIG. 5b shows a cumulative release curve of bulk Gel-DNPs with different pore-forming factor components (40%-80%); FIG. 5c shows a typical SEM image of a gel loaded with drug nanoparticles that are surface-modified with acrylate (Gel-DNPs-AC); and FIG. 5d shows a long-term cumulative release curve of bulk Gel-DNPs and bulk Gel-DNPs-AC with a pore-forming factor fraction of 70%.

FIG. 6a shows rheological curves showing the formation of hydrogels of Gel-DNPs and Gel-DNPs-AC under UV light induction, respectively; and FIG. 6b shows swelling and degradation properties of the two hydrogels.

FIG. 7a shows a schematic diagram of hydrogels of different shapes: Gel-DNPs-AC of bulk structure, thin-film Gel-DNPs-AC, and microhydrogel Gel-DNPs-AC, with a pore-forming factor fraction of 70%; and FIG. 7b shows a cumulative release curve of Gel-DNPs-AC of bulk structure, thin-film Gel-DNPs-AC, and microhydrogel Gel-DNPs-AC.

FIGS. 8a-8c show biocompatibility assessment of Gel-DNPs-AC; wherein FIG. 8a shows a schematic diagram of biocompatibility testing of hydrogels with mammalian cells via Transwell; FIG. 8b shows viability of B-SC-1 cells treated with Gel-DNPs-AC for 24 hours and 48 hours, with data reported as mean±SD (n=5); and FIG. 8c shows fluorescence imaging of live/dead BSC-1 cells stained with a kit after being treated with Gel-DNPs-AC for 48 hours, wherein live cells are stained with calcein AM (green) and dead cells are stained with PI (red), with a scale bar: 200 m.

FIGS. 9a-9d show a wound healing study in diabetic mice; wherein FIG. 9a shows a schematic diagram of an animal experimental design for testing the therapeutic effects of a composite hydrogel in normal and diabetic mouse models; FIG. 9b shows a representative image of wounds treated with or without the composite hydrogel for 14 days; FIG. 9c shows a graph of wound sizes at different time points, normalized to day 0; and FIG. 9d shows quantification of wound size changes within 14 days after injury; *P<0.05, **P<0.01, ***P<0.001.

FIGS. 10a-10c show evaluation of re-epithelialization and angiogenesis; wherein FIG. 10a shows immunofluorescence staining of cytokeratin 14 (CK14, red) in wounds on day 7 and day 14, with a scale bar of 200 m; FIG. 10b shows immunofluorescence staining of blood vessels (CD31, yellow) in a wounded area on day 7 and day 14, with a scale bar of 50 m; in all immunofluorescence staining images, cell nuclei are all stained with DAPI; and FIG. 10c shows quantification of vascular density; *P<0.05, **P<0.01, ***P<0.001.

FIGS. 11a-11c show evaluation of collagen regeneration and scarless healing (a drug-loaded hydrogel promotes scarless wound healing in diabetic wounds); wherein FIG. 11a shows H&E staining images of wounds after 7 and 14 days, with a scale bar of 200 m (black arrow for epidermis; and red arrow for skin appendages); FIG. 11b shows Picrosirius staining of wounded skin on day 14, with a scale bar of 100 m; and FIG. 11c shows quantification of total collagen deposition on day 14, *P<0.05, **P<0.01, ***P<0.001.

FIGS. 12a-12d show UV-Vis spectra of drug nanoparticles of F-127-encapsulated aspirin DNPs (FIG. 12a), DSPE-PEG-AC-encapsulated aspirin DNPs (FIG. 12b), DSPE-PEG-AC-encapsulated lidocaine DNPs (FIG. 12c), and DSPE-PEG-AC-encapsulated ropivacaine DNPs (FIG. 12d).

FIGS. 13a-13c show standard concentration curves for aspirin (FIG. 13a), ropivacaine (FIG. 13b), and lidocaine (FIG. 13c) established by UV-Vis spectrophotometry.

FIGS. 14a-14b show the shape of hydrogels with different PEG ratios before and after soaking; FIG. 14a shows freeze-dried drug-loaded hydrogels with 40%, 60%, 70%, 72%, 75%, 80% of PEG; FIG. 14b shows the disintegrated hydrogels with 70%-80% of PEG after soaking.

FIG. 15 shows FTIR spectrum of the prepolymer materials (PEG and PEGDA), amphiphilic polymers (F-127 and DSPE-PEG-AC), and both types of hydrogel systems post-polymerization.

FIGS. 16a-16b show scratch assays evaluating cell migration; FIG. 16a shows representative bright-field images of HaCaT and HUVEC scratch assays at 0 h and 24 h post-treatment. Scale bar: 200 m; FIG. 16b shows quantitative analysis of cell migration rates.

FIGS. 17a-17b show expression of inflammatory mediators in LPS-stimulated RAW264.7 macrophages; FIG. 17a shows Western blot analysis of inflammatory proteins (TNF-α and NF-κB) expression. β-actin is used as a loading control; FIG. 17b shows quantitative analysis of protein expression levels normalized to β-actin (n=3, *p<0.05, **p<0.01, ***p<0.001 vs. LPS group), and ELISA analysis of pro-inflammatory cytokine (PGE2) secretions in cell culture supernatants (n=3, *p<0.05, **p<0.01, ***p<0.001 vs. LPS group).

FIG. 18 shows body weight record of mice during the experiment.

### DETAILED DESCRIPTION

The present disclosure will be further explained in detail hereafter by specific examples. Unless otherwise specified, the raw materials, reagents or devices used in the examples and comparative examples are commercially available, or can be obtained by methods in the prior art. Unless otherwise specified, all experimental or testing methods are conventional methods in the art.

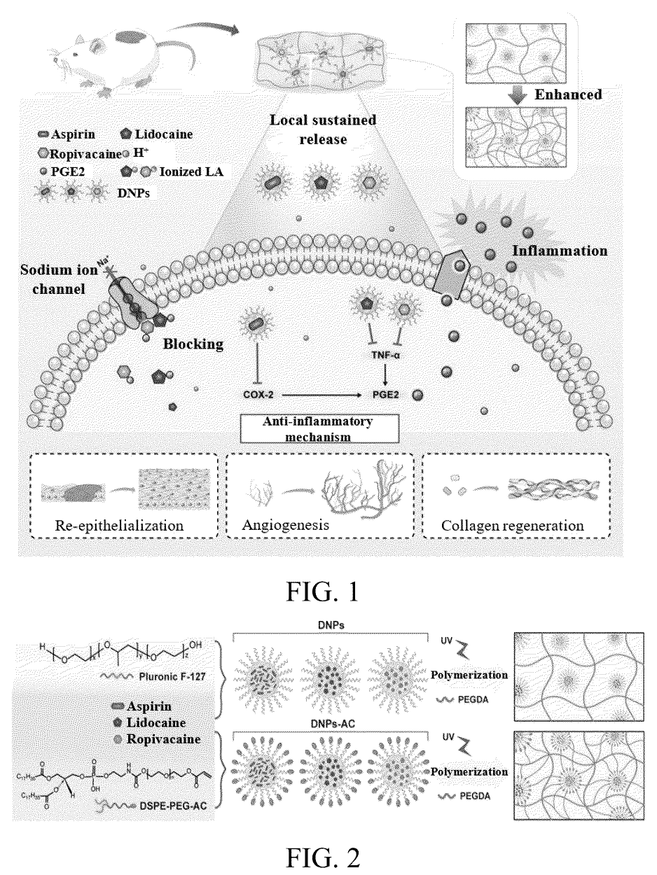

FIG. 1 illustrates an anti-inflammatory mechanism of a sustainable therapeutic platform. A topical hydrogel releases a cocktail of an anti-inflammatory drug and a local anesthetic onto a wound of a diabetic mouse. During crosslinking process, the hydrogel structure and the sustained release are enhanced by the surface properties of drug nanoparticles. Inside a cell, the combination of the drug molecules regulates signaling pathway and inhibits the secretion of PGE2, thereby achieving an anti-inflammatory effect.

### Example 1

In the present example, a drug nanoparticle was prepared, which was a mixture formulation of anti-inflammatory and local anesthetic nanoparticles. As shown in FIG. 2, active drugs (aspirin, ropivacaine, and lidocaine) were encapsulated by Pluronic F-127 and DSPE-PEG-AC amphiphilic polymers, respectively. The drug nanoparticle was prepared by a reprecipitation method. The specific process was as follows:

The organic drug molecules were each dissolved in a good-solvent methanol at a concentration of 1,000 ppm; and the polymer encapsulating agents were also dissolved in the same good solvent at the same concentration. A mixture of 200 μL of the drug solution (1,000 ppm) and 200 μL of the polymer solution (1,000 ppm) as prepared above was rapidly injected and dispersed into 10 mL of ultrapure water, to prepare a self-assembled drug nanoparticle mixture under a disturbance action of high-energy ultrasound (100 W, 100 s). The organic solvent was removed by purging with nitrogen, a small amount of aggregate was removed by filtration through a 0.22 m filter membrane, and the excess polymer encapsulating agent was removed by dialysis to obtain drug nanoparticles uniformly dispersed in an aqueous solution.

The drug nanoparticle solution was added dropwise onto a surface of a silicon wafer, which was dried and adhered to a copper platform, and then coated with carbon to increase the conductivity of organic particles. A PEGDA hydrogel sample was freeze-dried, and then torn open with a tweezer to observe the side cross-sectional morphology. The hydrogel sample was fixed on the copper platform and then coated with gold to increase conductivity for observation.

In the present example, Pluronic F-127 (referred to as F-127 for short) is a nonionic, surfactant polyol, which can promote the dissolution of hydrophobic drugs in physiological media to assemble DNPs. The amphiphilic polymer (DSPE-PEG-AC) is also a typical encapsulating agent, but its polymer molecule contains acrylate groups (green dots), which can assemble DNPs-acrylate (DNPs-AC) and can crosslink with the gel precursor PEGDA. The DSPE-PEG-AC used in the present example had a PEG segment with a molecular weight of approximately 2,000 Da (DSPE-PEG2000-Acrylate). PEGDA can then crosslink with DNPs-AC to enhance the pore structure of the hydrogel, thereby achieving sustained drug release.

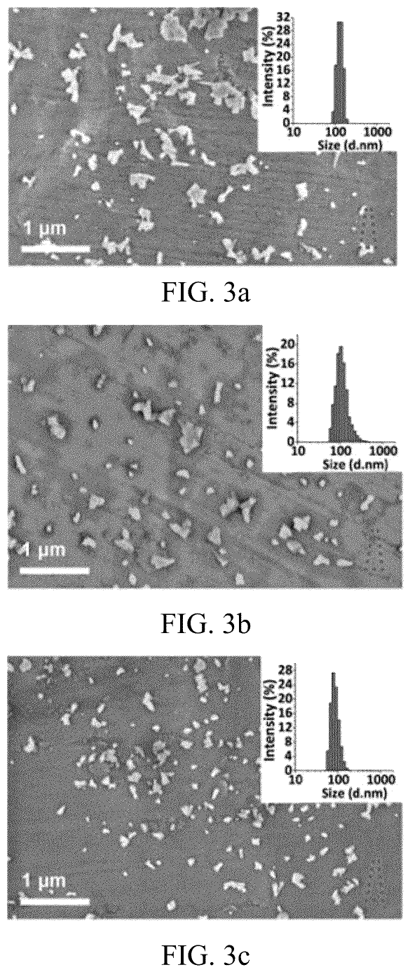

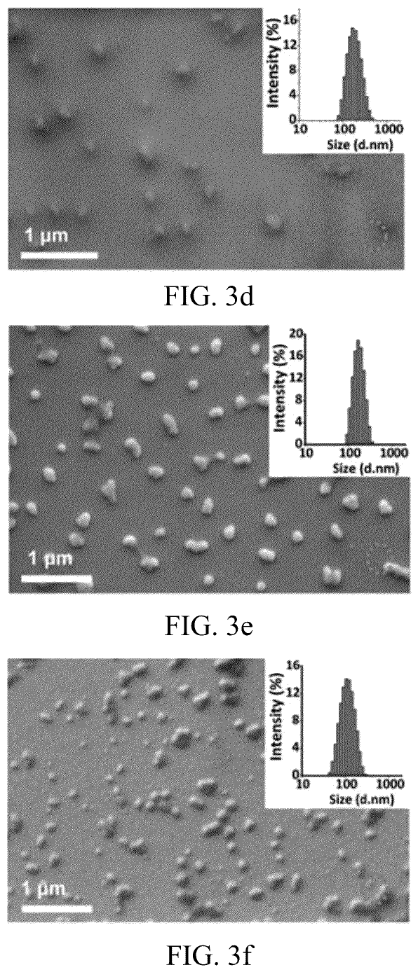

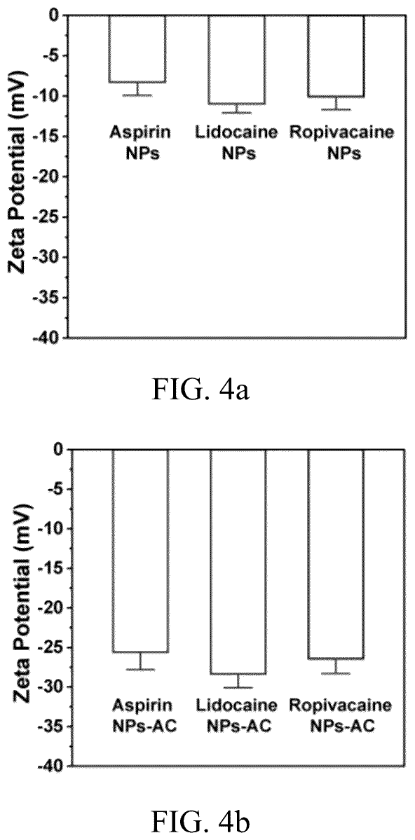

Aspirin, ropivacaine, and lidocaine were encapsulated into nanoparticles by Pluronic F-127 or DSPE-PEG-AC to achieve water solubility and stability. The DNPs of aspirin, lidocaine, and ropivacaine encapsulated by Pluronic F-127 were characterized by SEM and DLS (dynamic light scattering) measurements (FIGS. 3a, 3b, and 3c), indicating that their particle sizes were 122 nm, 105 nm, and 79 nm, respectively. These nanoparticles exhibited irregular shapes and low zeta potential values (FIG. 4a). In contrast, the DNPs-AC of aspirin, lidocaine, and ropivacaine encapsulated by the DSPE-PEG-AC polymer showed regular spheroidal shapes with particle sizes of 175 nm, 164 nm, and 101 nm, respectively (FIGS. 3d, 3e, and 3f). The DSPE-PEG-AC provided uniform assembly of the DNPs-AC and an outer layer distribution of acrylate, resulting in a zeta potential approximately three times that of the DNPs without acrylate (FIG. 4b). The larger zeta potential values led to more stable distribution of drug nanoparticles in water. Meanwhile, the surface acrylate could be crosslinked with PEGDA to promote stable loading of DNPs-AC in the hydrogel.

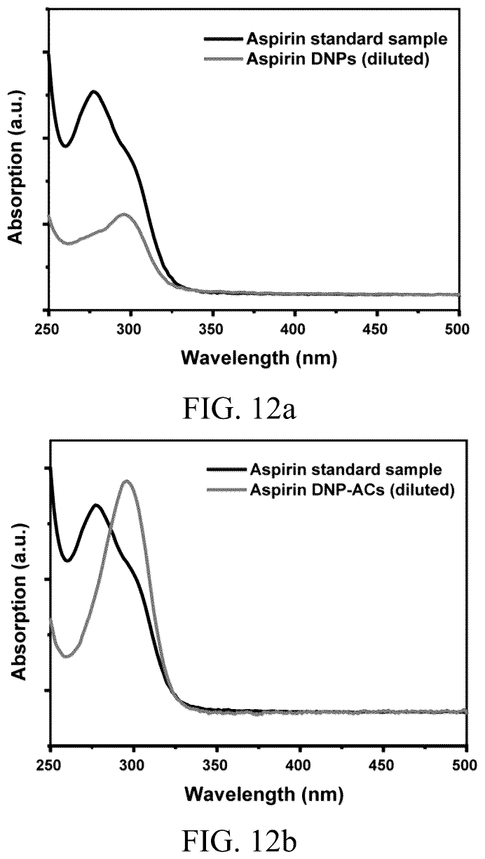

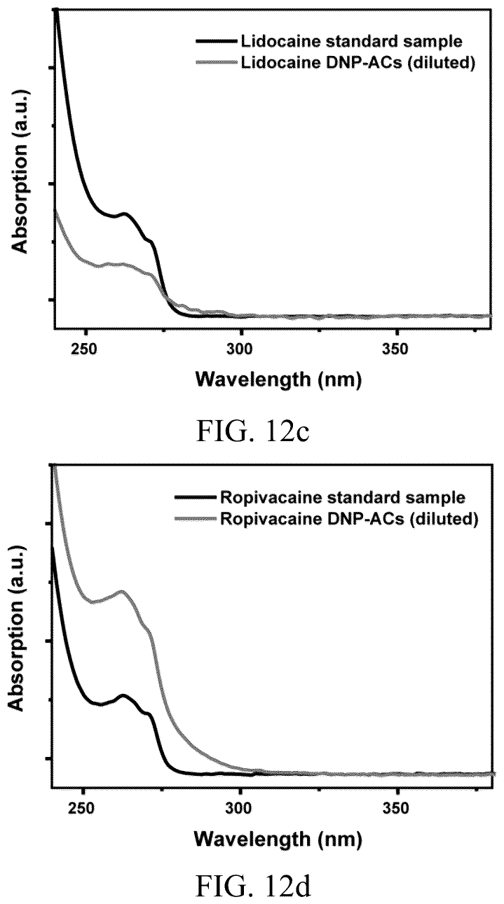

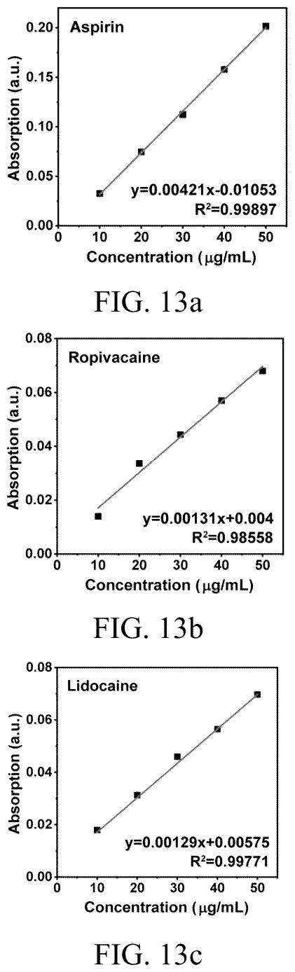

The UV-Vis absorption spectra of all drug nanoparticles were demonstrated in the FIGS. 12a-12d. Drug encapsulation efficiency (EE %) was calculated using the standard concentration curves for aspirin (FIG. 13a), ropivacaine (FIG. 13b), and lidocaine (FIG. 13c) established by UV-Vis spectrophotometry and ultrafiltration centrifugation. The F-127-encapsulated nanoparticles showed EE % of 79.56±0.25%, 82.59±0.14%, and 77.47±0.25% for aspirin, ropivacaine, and lidocaine, respectively. DSPE-PEG-AC-encapsulated formulations demonstrated enhanced encapsulation, achieving 82.86±0.18%, 85.29±0.26%, and 87.30±0.15% for aspirin, ropivacaine, and lidocaine, respectively. These results demonstrated that DSPE-PEG-AC encapsulation not only provided surface functionality but also achieved higher drug encapsulation efficiency compared to F-127 encapsulation across all three drug molecules.

### Example 2

In the present example, a drug-releasing hydrogel was prepared. A drug-loaded hydrogel was prepared using a cross-linkable PEGDA, porous PEG, and the DNPs solution prepared in Example 1. PEGDA and PEG were used for forming a hydrogel, which have been shown to have good biocompatibility, biodegradability, and immunological inertness. PEG has been used as a pore-forming agent to introduce porosity into the hydrogel, thereby enhancing accessibility of drug loading. The PEGDA used had a number average molecular weight of approximately 700 Da (PEGDA 700, Sigma-Aldrich). The pore-forming agent PEG had a molecular weight of approximately 600 Da (PEG 600, Sigma-Aldrich). The specific process was as follows.

A PEGDA hydrogel was prepared by in situ free radical polymerization. The prepolymer solution was composed of PEG and a reactive mixture. The PEG content was varied at 40 wt %, 60 wt %, 70 wt %, 75 wt %, or 80 wt % of the total hydrogel weight. The remaining portion (60 wt %, 40 wt %, 30 wt %, 25 wt %, or 20 wt %, respectively) consisted of a reactive mixture containing 33 wt % PEGDA, 59 wt % drug nanoparticle solution, and 8 wt % initiator. The prepolymer mixture was added into a mold and cured for 1 minute under an ultraviolet lamp (365 nm, 120 mJ/cm²). The acrylate on the surface of the drug nanoparticle encapsulated by DSPE-PEG-AC could be crosslinked with PEGDA to synthesize a more stable hydrogel framework loaded with drug nanoparticles.

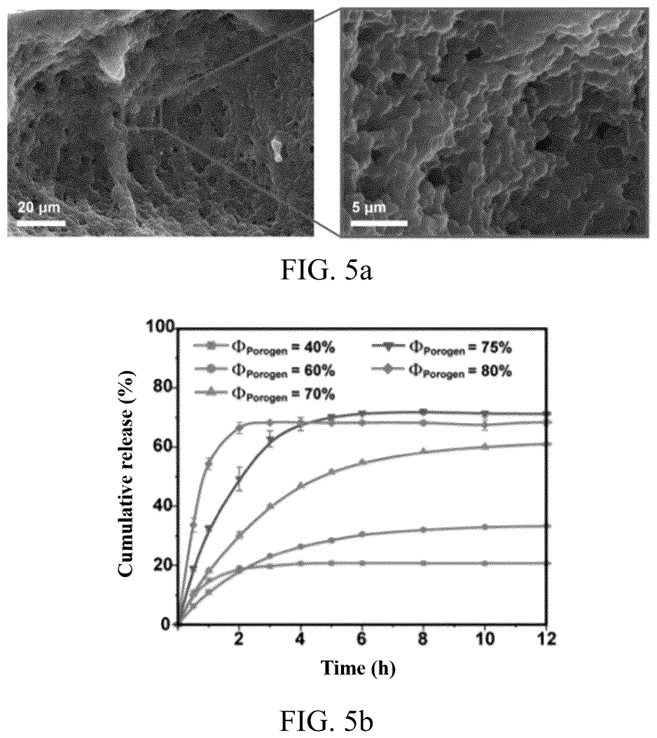

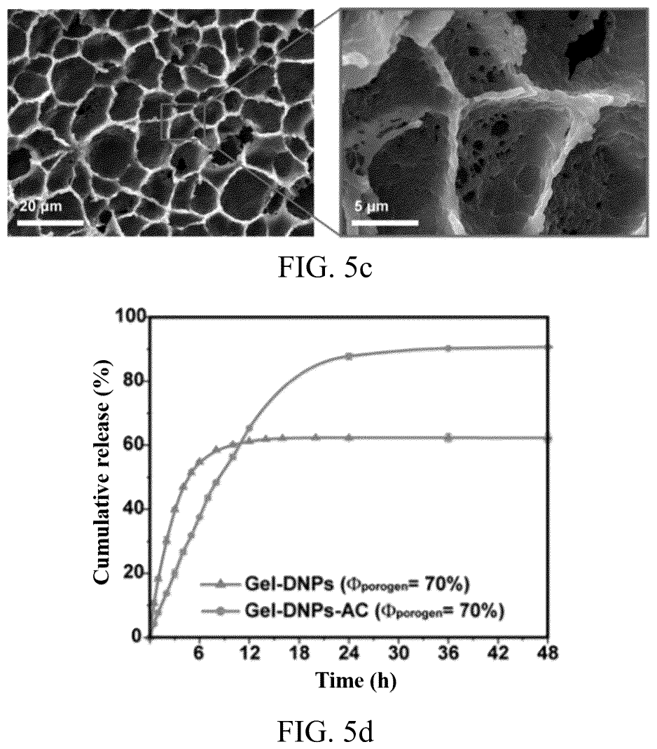

FIG. 5a shows the morphology of a DNP-loaded hydrogel (Gel-DNPs), which was prepared by using a precursor formulation of 10 wt % of PEGDA, 70 wt % of PEG, and 17.6 wt % of a DNPs solution. The PEG pore-forming agent provided a hydrogel structure with cave-like channels, which facilitated the loading of the DNPs solution. Therefore, drug release characteristics of the hydrogel could be regulated by adjusting the amount of the pore-forming agent to meet the demands of different application scenarios.

The effect of the porous PEG component on the drug release characteristics was studied by placing the Gel-DNPs in a PBS solution shaken at a constant temperature of 37° C. The specific process was as follows.

The DNPs-loaded hydrogel was placed into PBS in a flat-bottom centrifuge tube, and shaken with a shaker at 60 rpm in an incubator at 37° C. to simulate the in vivo environment. 1 mL of a drug release solution was periodically pipetted from the centrifuge tube for UV-visible light testing. The PBS was replaced with the same volume of fresh PBS at each interval to maintain perfect sedimentation conditions. The soaking solution was analyzed by an UV-visible spectrophotometer at the wavelength of characteristic absorption peak of the drug. The cumulative release and absorption of the drug-releasing hydrogel were calculated according to the following equation:

$$Cumulative Abs = {V}_{0} {A}_{n} + \sum V {A}_{n - 1}$$

wherein Aₙ and Aₙ₋₁ were the drug absorption of the n-th and (n-1)-th sampling respectively, V₀ was the initial volume of the drug-releasing medium, and V was the sampling volume.

The drug-releasing hydrogels were prepared by varying the porous PEG fraction from 20 wt % to 90 wt %. It was observed that when below the Φ_{pore-forming agent}=70%, the release duration was prolonged with the increase of the fraction of pore-forming agent (FIG. 5b). At the PEG fraction of 70%, the duration for complete drug release reached up to 12 hours. However, the increase in porosity was accompanied by a decrease in structural strength. When the Φ_{pore-forming agent}=70% was exceeded, the duration for complete release began to decrease. For example, the duration for complete release at the Φ_{pore-forming agent}=75% was 6 hours.

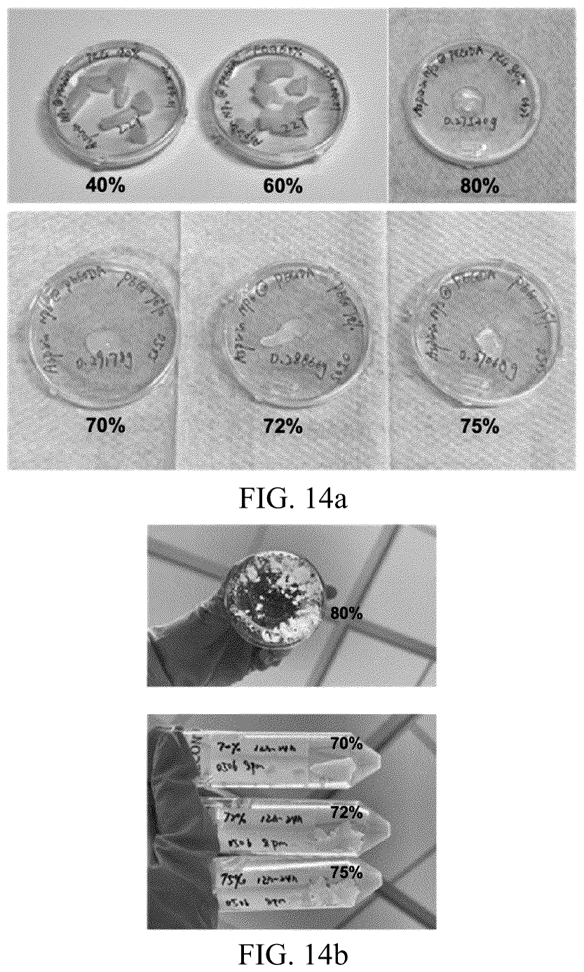

It should be noted that, excessive addition of the pore-forming factor led to an increase in the surface area and structural instability of the hydrogel. This would cause the hydrogel to collapse rapidly in body fluids, leading to rapid drug release. In the in vivo environmental simulation, the hydrogel samples were soaked in PBS in flat-bottom tubes, and kept at 37° C. in the incubator with a shaker at 60 RPM to mimic in vivo environment. After 12 h of soaking, the hydrogels with 70% PEG were still in shape, while the hydrogels with 72% PEG were disintegrated in 6 h and the hydrogels with 75% and 80% PEG were disintegrated between 1-2 h (FIGS. 14a-14b). The Gel-DNPs with the Φ_{pore-forming agent}=80% (PEG as pore-forming agent) disintegrated after 2 hours. Thus, this type of hydrogel is suitable for application scenarios where rapid drug release and high concentrations of local drug release are required.

For the treatment of chronic wounds, in order to maintain the steady-state concentration, the formulation of gel precursor was controlled at Φ_{pore-forming agent}=70%.

The surface acrylate groups of drug nanoparticles were crucial for the formation of regular pore structures in the hydrogel. The hydrogel was crosslinked with DNPs-AC (Gel-DNPs-AC), and the precursor consisted of 10 wt % of PEGDA, 70 wt % of PEG, and 17.6 wt % of the DNPs-AC solution. As shown in FIG. 5c, the Gel-DNPs-AC platform exhibited a well-defined pore structure compared with the Gel-DNPs, indicating that DNPs-AC played an important role in the formation of regular pores. The magnified SEM image showed that the pore size in the Gel-DNPs-AC varied in a range of tens of micrometers. Regular pores and channels facilitated the release of drugs from the Gel-DNPs-AC hydrogel. By comparing the Gel-DNPs and the Gel-DNPs-AC with the same PEG fraction (Φ_{pore-forming agent}=70%), the influence of the crosslinking between the nano-drug and the hydrogel on the drug release characteristics was studied. Obviously, the Gel-DNPs-AC exhibited sustained release performance. Gel-DNPs-AC showed a release duration exceeding 24 hours with a cumulative drug release amount higher than 90% (FIG. 5d), indicating that the crosslinking between the DNPs-AC and the hydrogel resulted in long-term sustainable and complete release compared with a gel physically loaded with DNPs. The crosslinking between the DNPs-AC and PEGDA might create a large number of stable attachment sites for nano-drugs on a hydrogel scaffold, enhancing the drug loading capacity of the composite hydrogel.

Testing of swelling ratio and dynamic rheology of the drug-releasing hydrogel:

The swelling ratio of the hydrogel in water was determined using a gravimetric method. The hydrogel sample was soaked in water until it reached a constant weight, and was then quickly removed from the water. The water on the surface of the hydrogel was removed with filter paper, and then the hydrogel was weighed to obtain an equilibrium swelling mass. The equilibrium swelling ratio (ESR) of the hydrogel was calculated according to the following equation:

$$ESR = \frac{{W}_{s} - {W}_{d}}{{W}_{d}} \times 100 \%$$

wherein Wₛ was the weight of the hydrogel after swelling equilibrium; and W_{d} was the dry weight of the hydrogel (after freeze-drying).

The degradation rate was evaluated by weighing the freeze-dried sample after soaking. The hydrogel sample was soaked in water until a constant weight was reached, and then taken out and freeze-dried. After the water was removed, the sample was weighed to obtain a degradation mass. The degradation rate (DR) of the hydrogel was calculated according to the following equation:

$$DR = \frac{{W}_{d} - {W}_{de}}{{W}_{d}} \times 100 \%$$

wherein W_{d} was the dry weight of the hydrogel before soaking; and W_{de} was the dry weight of the hydrogel after soaking. The dynamic rheological performance of the hydrogel was tested using a rotational rheometer (HAAKE MARS III). Under UV irradiation, the storage modulus and loss modulus were measured as a function of time and frequency.

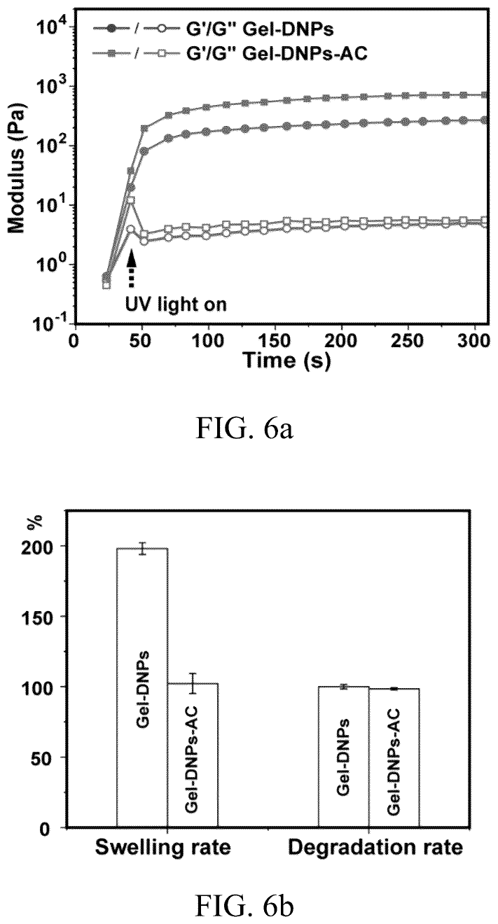

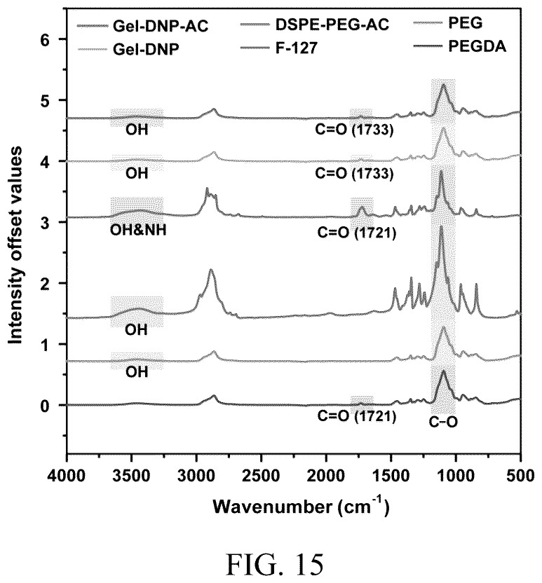

The dynamic rheological properties were analyzed by time sweep testing (FIG. 6a). After light treatment, the storage modulus (G′) was greater than the loss modulus (G″). Upon UV irradiation, the two gel precursors with two types of DNPs changed from liquid to solid within seconds and immediately exhibited elastic behaviors. The storage modulus of Gel-DNPs-AC was greater than that of Gel-DNPs, indicating that participation of the DNPs-AC in the hydrogel framework enhanced the mechanical performance of the hydrogel. The improved mechanical performance was consistent with the well-defined pore structure of the Gel-DNPs-AC sample. FTIR spectroscopic analysis was performed to verify the polymerization of Gel-DNPs and Gel-DNPs-AC systems. The characteristic shift of carbonyl bands in the FTIR spectra (FIG. 15) provided evidence for successful polymerization, demonstrating the transformation from prepolymers to their respective hydrogel networks. The swelling ratios and degradation rates of the two types of structured hydrogels were evaluated in a PBS buffer (pH 7.4). As shown in FIG. 6b, the two types of hydrogels degraded by 97% after being soaked in PBS for one week, indicating that the hydrogels had good biodegradability. The water absorption capacity of the Gel-DNPs-AC was significantly lower than that of the Gel-DNPs, indicating that the Gel-DNPs-AC had a rigid framework with a pore structure. The acrylate surface modification on the DNPs-AC increased the crosslinking density of the composite hydrogel and led to the formation of a regular pore structure, thereby limiting the swelling performance of the hydrogel. The swelling characteristic of the hydrogel was highly indicative of the absorption of the wound exudate by the wound dressing. The low swelling ratio of the hydrogel was beneficial for maintaining mechanical strength for skin therapeutic applications.

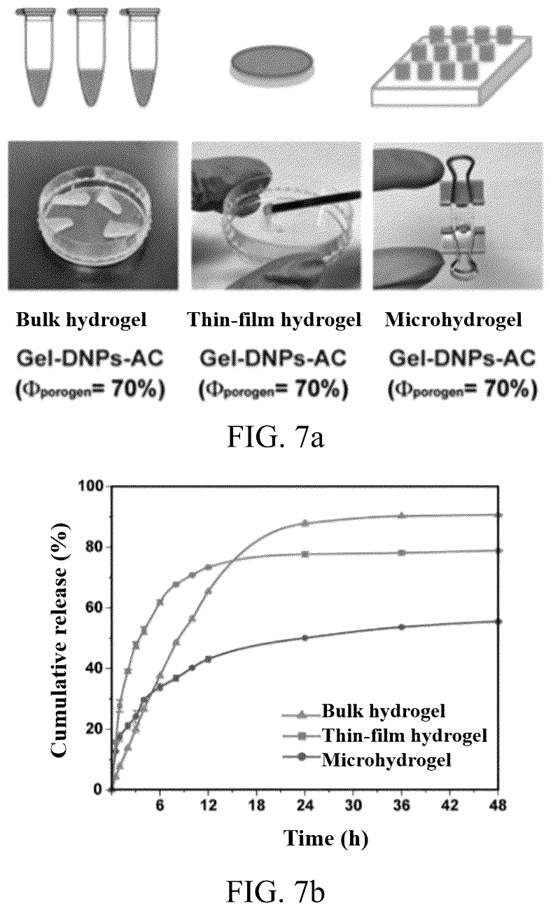

The present example also studied the effect of the hydrogel shape on drug release. As shown in FIG. 7a, the precursor solution of 70% of the pore-forming agent and the DNPs-AC was added into a mold and cured under UV light to form a bulk hydrogel, a film hydrogel, and a microhydrogel. The release rate of the microhydrogel was the slowest among the 3 shapes, which might be related to the larger specific surface area of the microhydrogel and the different mechanical performances of oscillation in fluids. The film hydrogel suitable for skin treatment showed release characteristics comparable to those of the bulk hydrogel, with a duration for complete release of less than 24 hours (FIG. 7b).

### Example 3

In the present example, the biocompatibility of the Gel-DNPs-AC was evaluated by MTT and cell staining assay. The specific process was as follows.

Mammalian cell BS-C-1 cells were cultured in Dulbecco's Modified Eagle Medium containing 10% fetal bovine serum and 1% penicillin/streptomycin in a humid environment of 37° C. and 5% CO₂. The cell viability and cytotoxicity of the Gel-DNPs-AC in vitro were evaluated by MTT test and calcein AM test. 1×10⁴ BS-C-1 cells were inoculated into a 24-well cell culture plate, and cultured overnight to allow adherence. The drug-releasing hydrogel was then cut into cylindrical shapes (6 mm*2 mm), placed into Transwell inserts, positioned above the cells in proper order to allow the hydrogel to be soaked and release the drug. The cells were incubated in an environment at 37° C. and 5% CO₂ for 24 hours or 48 hours. After incubation, each well was added with MTT and incubated for another 4 hours. After addition of dimethyl sulfoxide, the absorbance (OD570) of each well was measured using a microplate reader (BioTek Cytation 3), and the background was subtracted. Cell viability was calculated according to the following equation:

$$Cell viability ( \% ) = \overline{{A}_{t}} / {A}_{c}$$

wherein Āₜ was the average absorption value of the treatment group, and A_{c} was the average absorption value of the control group.

The cell compatibility of the Gel-DNPs-AC was tested by a live/dead cell activity detection kit. Cells were inoculated according to the aforementioned method, and co-incubated with the hydrogel soaked in Transwell. Then, a calcein AM solution and a propidium iodide (PI) solution were added and incubated for 30 minutes and 10 minutes, respectively. The samples were washed with PBS for three times, and observed by confocal microscopy.

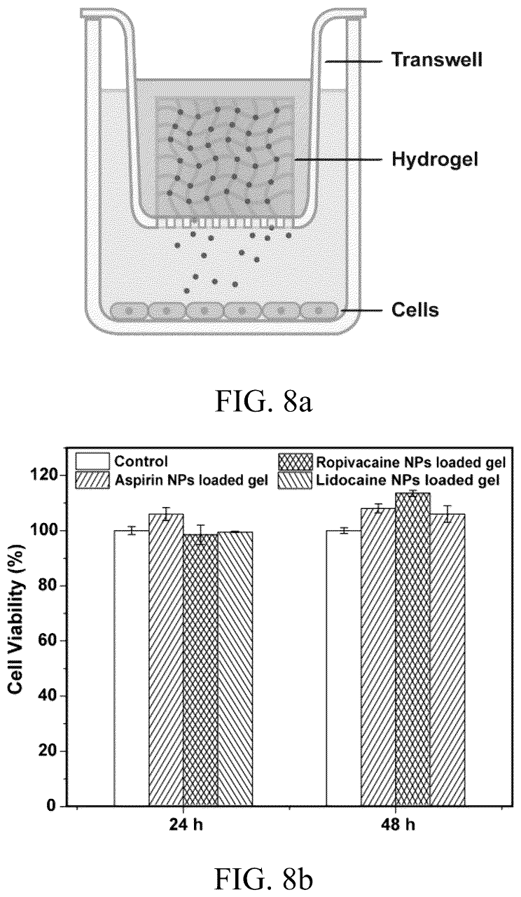

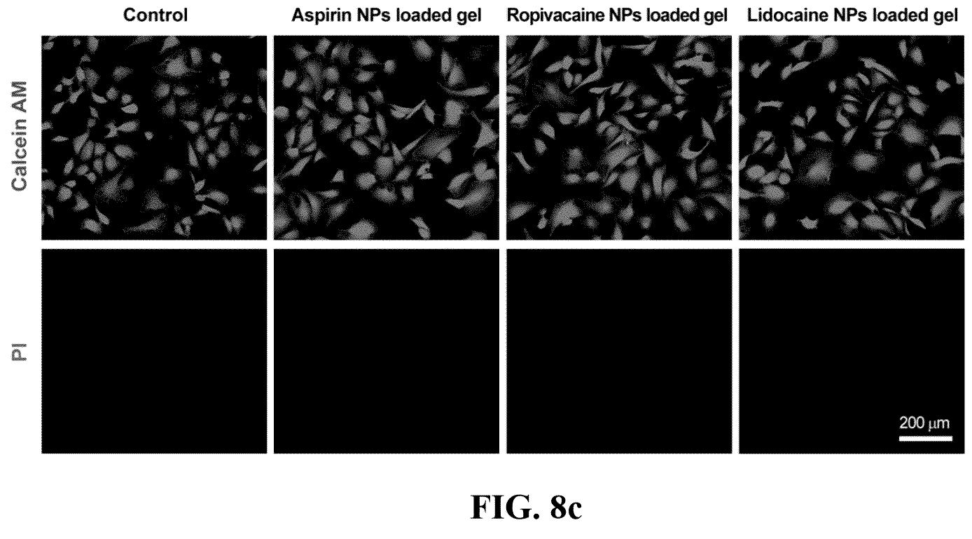

Three pieces of DNPs-AC gels, were studied using the aforementioned Transwell apparatus to evaluate drug release or leaching of monomer residues. Mammalian cells (BS-C-1) were treated with the hydrogels placed in the Transwell inserts (FIG. 8a). All the Gel-DNPs-ACs showed no significant cytotoxicity, as indicated by approximately 100% cell viability after 48 hours of incubation (FIG. 8b). Fluorescence staining showed that cells treated with the hydrogels containing the three types of DNPs-AC were similar to those of the control group (FIG. 8c), as evidenced by the bright fluorescence of live cells (calcein AM) and the absence of fluorescence of translocated cells (PI). These observations further confirmed the good biocompatibility of the Gel-DNPs-AC.

### Example 4

The present example assessed the impact of the hydrogel formulations of multiple drug combinations. The specific process was as follows.

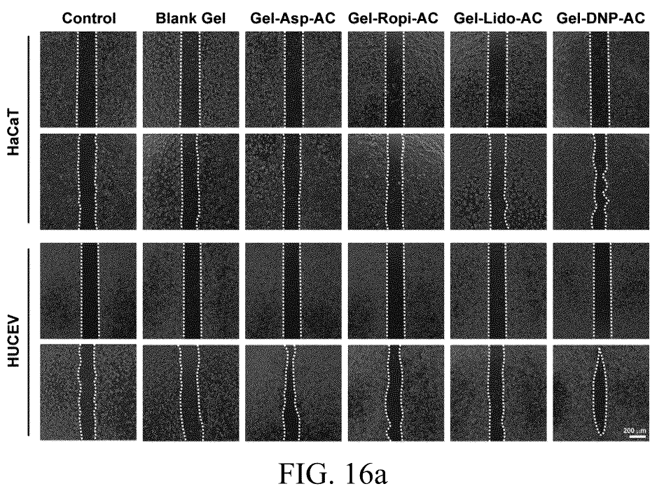

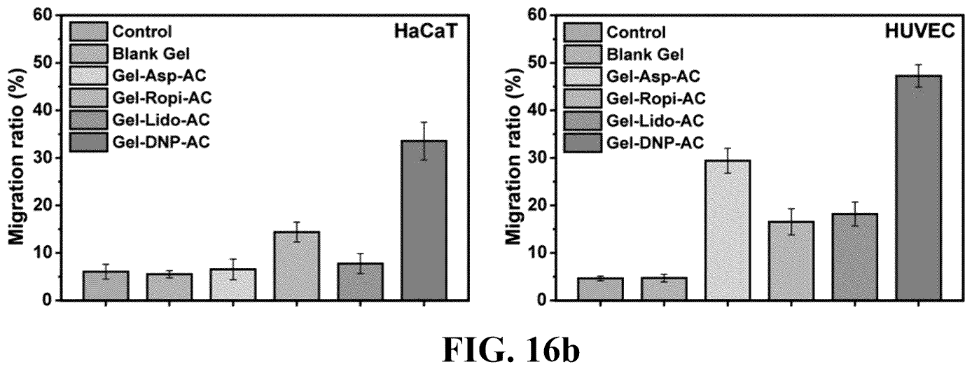

Cell migration by scratch assay. The evaluation of hydrogels loaded with individual drug nanoparticles (aspirin, ropivacaine, and lidocaine) and assessment of hydrogels simultaneously loaded with all three drug nanoparticles were conducted. Briefly, cells were seeded in 12-well plates and allowed to reach confluence overnight. After creating uniform scratches using a cell scratcher, the cells were treated with hydrogel extracts from different groups. Cell migration was monitored and photographed using microscopy over a 24 h period. The experiments were performed on two cell lines: human keratinocytes (HaCaT) and human umbilical vein endothelial cells (HUVEC). The results were shown in FIGS. 16a-16b. For HaCaT cells, among single-drug formulations, ropivacaine NPs-loaded hydrogels demonstrated the highest promotion of cell migration; For HUVEC, aspirin NPs-loaded hydrogels showed the most significant enhancement in cell migration among single-drug formulations. In both cell lines, hydrogels simultaneously loaded with all three drug nanoparticles (Gel-DNPs-AC) exhibited the highest overall promotion of cell migration. These results suggested that different cell types may have varying uptake and utilization patterns for these drugs. This observation underscored the importance of multi-drug combinations in addressing the complex wound environment. The superior performance of Gel-DNPs-AC in both cell types indicated a synergistic effect, potentially beneficial for wound healing. This finding supported the approach of using a multi-drug delivery system for enhanced wound healing outcomes.

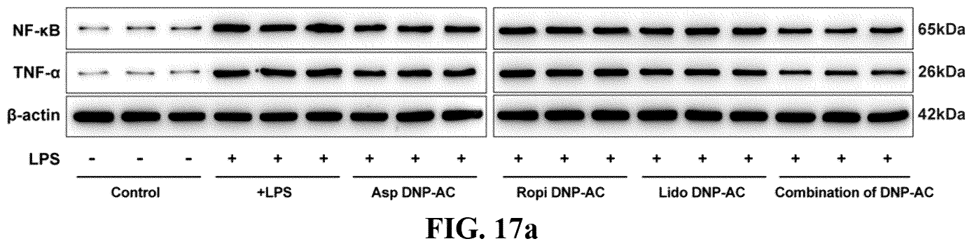

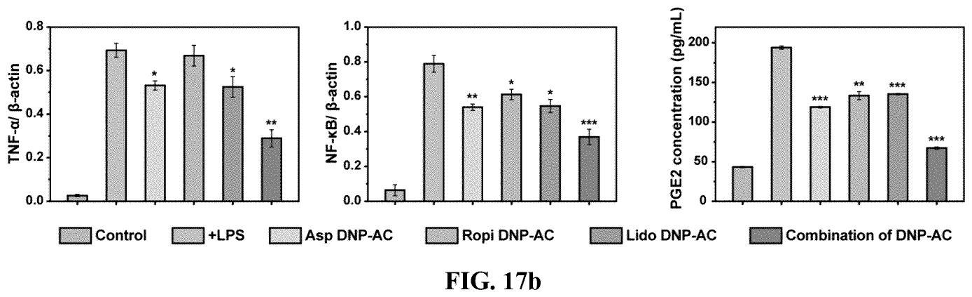

Drug effects on cellular inflammatory pathways. Western blot and ELISA experiments were conducted. Lipopolysaccharide (LPS) was used to induce inflammation in the cells and examined the individual and combined effects of three drug nanoparticles (DNPs) on the secretion of inflammatory factors, including TNF-α, NF-κB, and PGE2. RAW264.7 macrophages were stimulated with lipopolysaccharide (LPS) and simultaneously treated with different DNPs formulations. After 4 h of incubation, both cells and culture supernatants were harvested for Western blot and enzyme-linked immunosorbent assay (ELISA) analyses to evaluate the expression and secretion of inflammatory mediators. Through Western blotting and ELISA assays, it was confirmed that the combination of the three DNPs demonstrated the most effective inhibition of the inflammatory pathways (FIGS. 17a-17b).

### Example 5

The present example evaluated the efficacy of the Gel-DNPs-AC in wound healing in diabetic mice. The specific process was as follows.

Diabetic mice were induced by a streptozotocin (STZ) method. STZ was dissolved in citric acid and sodium citrate buffer, and each mouse was intraperitoneally injected with STZ (180 mg/kg) to induce diabetes. The blood glucose levels of the mice were measured after fasting overnight. After 10 days of injection, the blood glucose levels were stable to be more than 20 mmol/L, indicating that the modeling was successful. For each one of normal and diabetic mice, a circular wound with a diameter of 1 cm was created on the depilated dorsal skin for subsequent treatment. Diabetic mice were randomly divided into three groups (n=5). On days 0, 2, 4, 6, 8, and 10 after wound establishment, the wounds were treated with a blank hydrogel and the Gel-DNPs-AC, respectively. The mice were sacrificed on days 7 and 14, and skin tissues from the dorsal wound sites were collected. The tissues were fixed with 4% paraformaldehyde, embedded in paraffin, and subjected to hematoxylin-eosin (H&E) staining, Picrosirius staining, and immunofluorescence staining of CK14 and CD31.

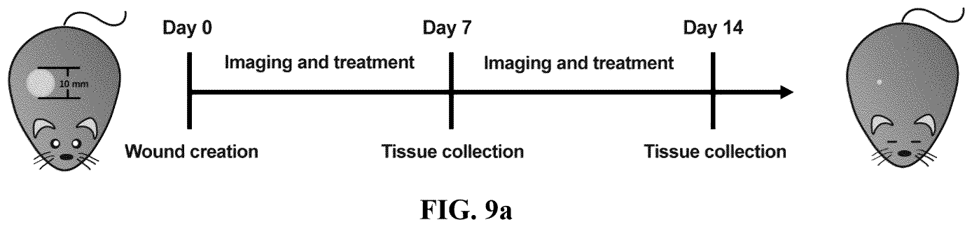

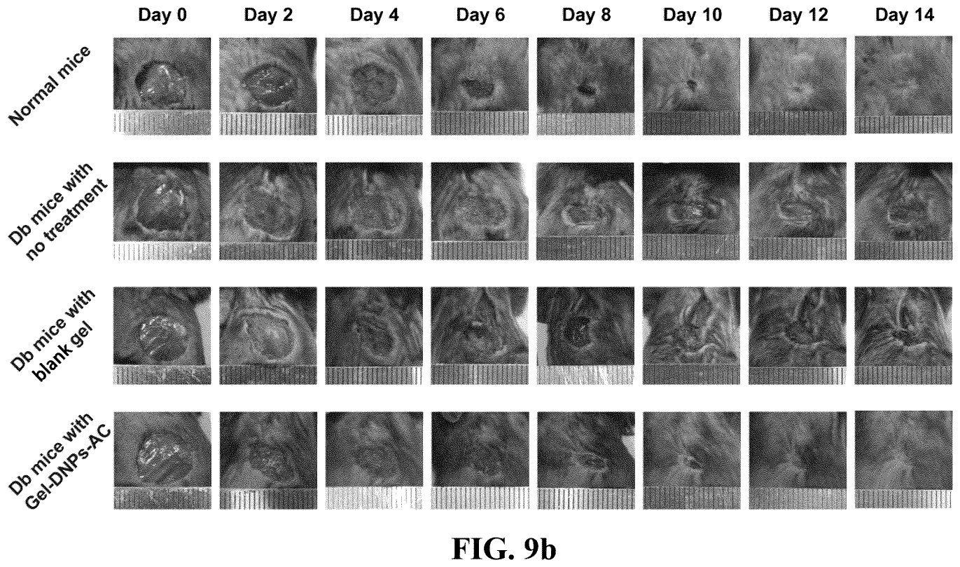

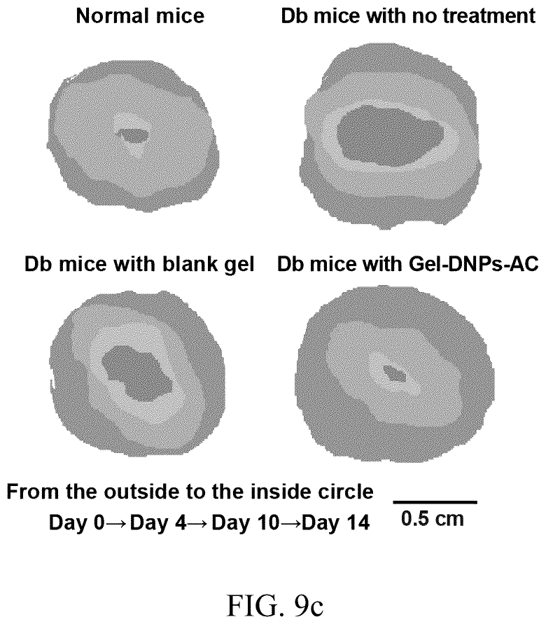

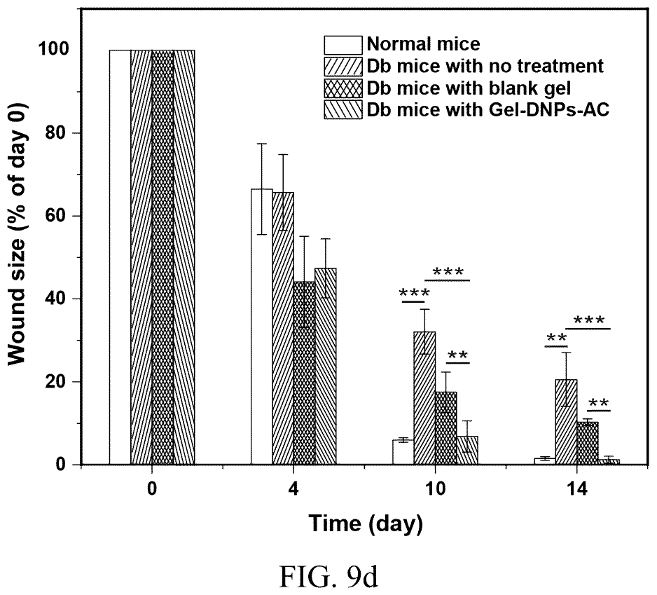

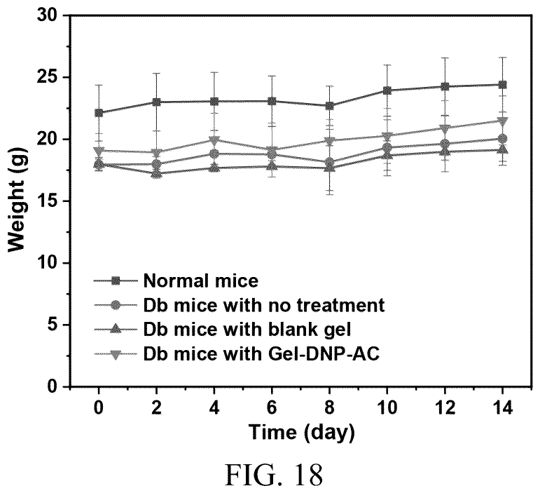

The hydrogel significantly accelerated wound closure compared with the control group (FIGS. 9a-9d). Diabetic wound healing was explored by applying Gel-DNPs-AC gel patches on the excisional wounds of diabetic mice (FIG. 9a). The anti-inflammatory drug-loaded DNPs-AC and local anesthetic-loaded DNPs-AC were incorporated into the composite hydrogel by crosslinking for cocktail therapy. Each gel patch contained a mixture of 40 μg of aspirin, 30 μg of ropivacaine, and 30 μg of lidocaine. As shown in FIG. 9b, the wounds of diabetic (Db) mice treated with the Gel-DNPs-AC exhibited a higher closure rate than those of untreated mice and the blank gel group. By day 14, the wounds of diabetic mice treated with the Gel-DNPs-AC were completely closed compared with those of the control group (FIG. 9d). Body weight analysis throughout the experiment indicated that the Gel-DNPs-AC hydrogel system had no adverse effects on mouse health (FIG. 18). The wounds of the Gel-DNPs-AC treatment group recovered and closed well, healing to 1.16% of the original wound size, which showed similar or better wound closure rates in the same rodent model (STZ-induced diabetic mice), as compared to those in previous studies using anti-inflammatory drugs to treat chronic wounds. Within the first four days, the wound area of the Gel-DNPs-AC group closed faster than that of the normal mouse group (FIG. 9c). These results suggested that the porous hydrogel promoted wound closure in the initial stage, while the Gel-DNPs-AC aided diabetic wounds to recover as effectively as normal wounds.

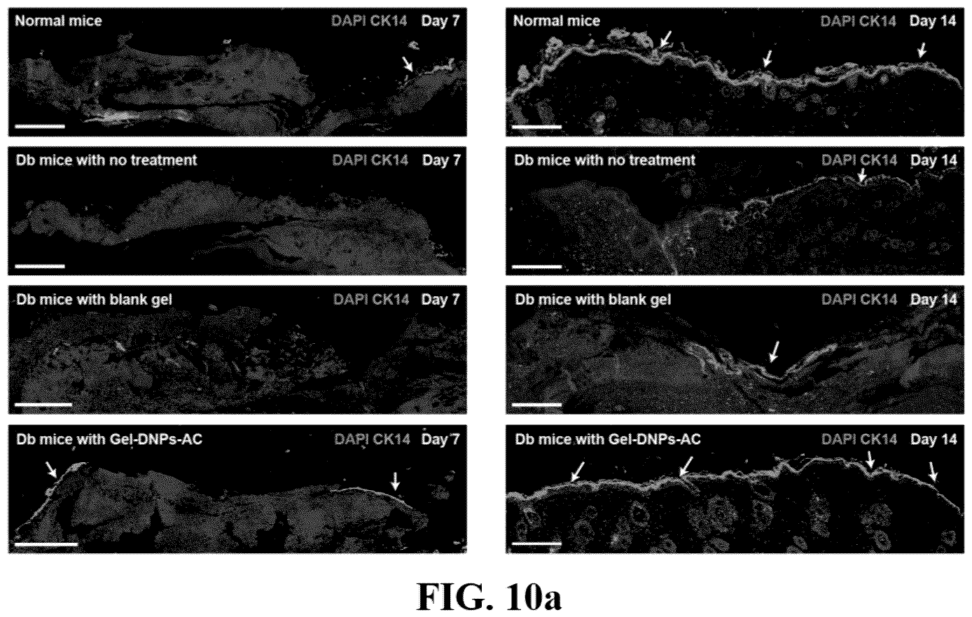

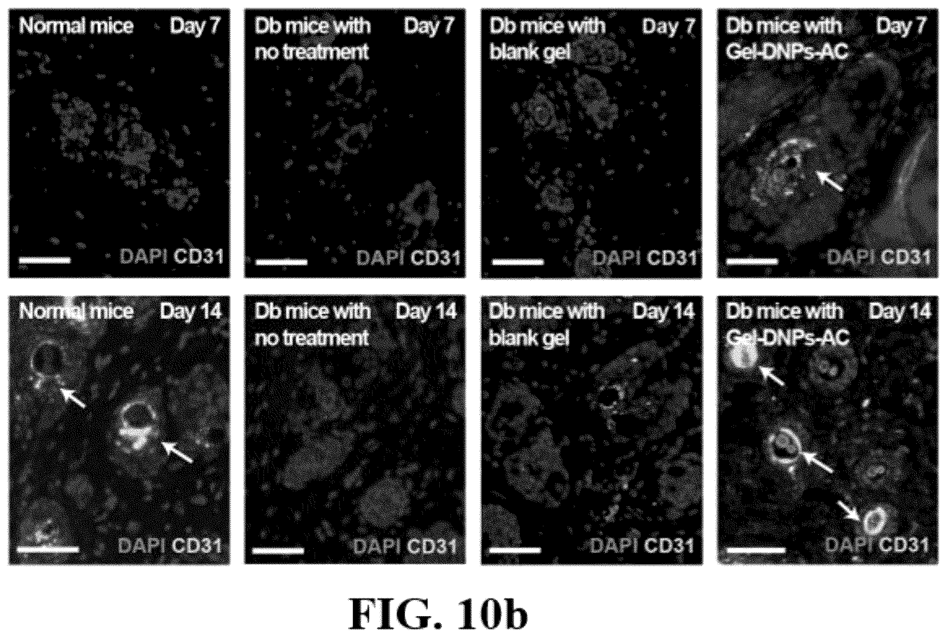

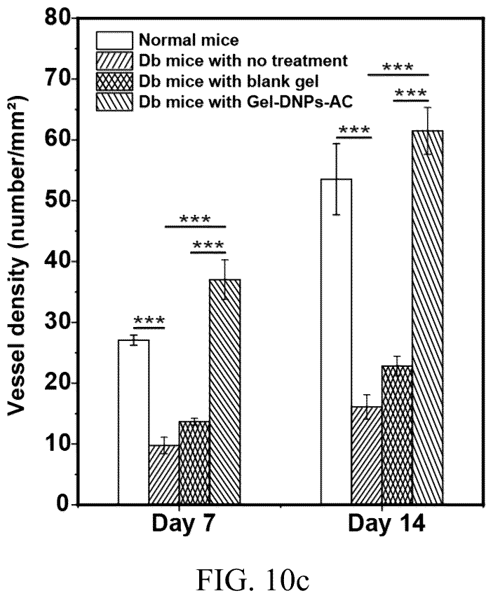

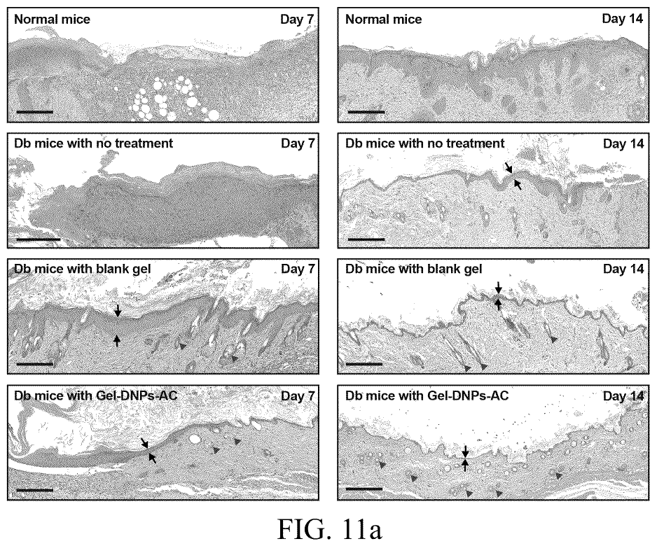

Evaluation of re-epithelialization and angiogenesis: histological analysis showed that re-epithelialization and angiogenesis of wounds treated with the Gel-DNPs-AC were enhanced (FIGS. 10a-10c). Tissue sections collected on day 7 and day 14 were stained with cytokeratin 14 (CK14, a marker of basal keratinocytes) to evaluate the re-epithelialization rate of the diabetic wound sites. On day 7, i.e., the midpoint of the healing period, half of the wound gaps were surrounded by the basal keratinocyte layer (CK14⁺) in the Gel-DNPs-AC group. In contrast, the migration of keratinocytes in other control groups were significantly slower than that in the treatment group (FIG. 10a). On day 14, the migration of basal keratinocytes was fully completed in both the Gel-DNPs-AC group and the normal mouse group. In the Gel-DNPs-AC group, clearer stratified epithelium was observed compared with that in normal mice without the gel. In contrast, in the untreated group and in the diabetic mice treated with the gel alone but without drug loading, the keratinocyte layer did not close, while migration, proliferation, and differentiation were still ongoing. These observations demonstrated that the sustained anti-inflammatory and pain-relieving effects of the Gel-DNPs-AC effectively accelerated keratinocyte migration, thereby promoting re-epithelialization of the diabetic wounds. In order to evaluate angiogenesis in the diabetic wounds, a platelet endothelial cell adhesion molecule-1 (CD31⁺) marker was used for quantifying the capillary densities in the middle stage (day 7) and the last stage (day 14) of wound healing. At these two stages, the capillary density (CD31⁺) in the Gel-DNPs-AC group was significantly higher than those in other control groups (FIGS. 10b and 10c, P<0.001). The results showed that the composite Gel-DNPs-AC promoted rapid angiogenesis and aided chronic wounds transition from a prolonged inflammatory phase to a proliferative phase.

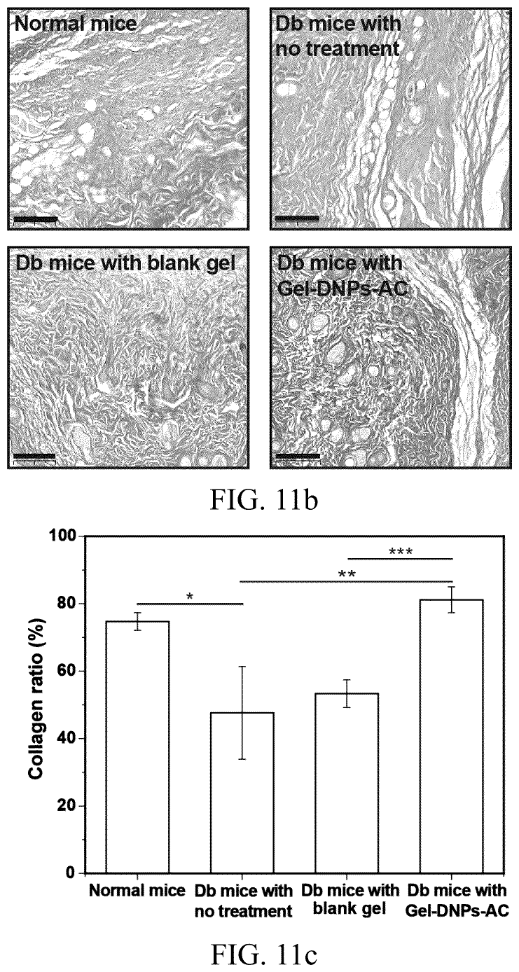

Evaluation of collagen regeneration and scarless wound healing: histological staining was used for evaluating collagen deposition and scar formation. The Gel-DNPs-AC promoted collagen regeneration and resulted in minimal scarring (FIGS. 11a-11c). To evaluate scarless skin repair at the diabetic wound sites, epidermal thickness was characterized by hematoxylin-eosin (H&E) staining. H&E staining demonstrated an inflammatory cell status, fibroblast proliferation, and neovascularization in the wound (FIG. 11a). On day 7, both the normal mouse group and the untreated diabetic mice showed a large amount of inflammatory cell infiltration. In contrast, the pure gel group and the Gel-DNPs-AC group exhibited fewer inflammatory cells, and some skin appendages (triangle arrows) regenerated, indicating that the PEGDA hydrogel contributed to the rapid functional recovery of skin tissues. On day 14, a large number of skin appendages and hair follicles were formed in the hydrogel treatment group, while far fewer skin appendages and hair follicles were observed in other control groups. Among the four groups, the Gel-DNPs-AC group exhibited the most effective regeneration, with regular arrangement of collagen fibers and skin appendages, and the thinnest neo-epidermis (black arrows) compared with those of all other control groups. This suggested that the nanodrug-hydrogel composite sustainable therapeutic platform could regulate the infiltration of inflammatory cells in the diabetic wounds, promoted the regeneration of skin tissues and the proliferation of fibroblasts, and facilitated scarless healing. Collagen regeneration, a key indicator of wound healing, was evaluated during the Gel-DNPs-AC treatment. The collagen was stained red and other tissues were stained yellow with Picrosirius dye. The density and depth of the two colors were compared to determine the collagen contents of the wound sites. As shown in FIGS. 11a-11c, in the Gel-DNPs-AC group, a large amount of collagen with a high-density of red area was regenerated at the wound site. On day 14, the three control groups had less red collagen (accounting for 81% of the total healing area, P<0.01) compared with the collagen content in the Gel-DNPs-AC group (accounting for 81% of the total healing area, P<0.01) (FIGS. 11b and 11c). Collagen regeneration was neat and flat, revealing that the enhanced wound treatment method of the present disclosure facilitated scarless wound healing.

Dental treatment fillings: as mentioned above, the shape of the drug-loaded hydrogel platform was malleable, and the release effects of different shapes had been studied. Therefore, this hydrogel drug-loaded platform could be used as a wound filler in dental treatment to provide sustained release of an active pharmaceutical ingredient in the affected area. It was characterized by good biocompatibility, biodegradability, no need for surgical resection, and sufficient softness, and thus was suitable for such treatment scenarios.

Usage as a postoperative filler: as mentioned above, the shape of the drug-loaded hydrogel platform was malleable, and the release effects of different shapes had been studied. Therefore, this hydrogel drug-loaded platform could be used as a postoperative wound filler to sustainably release an active pharmaceutical ingredient in the affected area. It had good biocompatibility and biodegradability. Compared with a traditional gauze filling method, it did not require surgical removal, and thus was suitable for such treatment scenarios.

The above examples are preferred embodiments of the present disclosure. However, the embodiments of the present disclosure are not limited by the above examples. Any other change, modification, substitution, combination, and simplification made without departing from the essence and principle of the present disclosure should be an equivalent replacement manner, and all are included in a claimed scope of the present disclosure.

What is claimed is:

1. A drug-releasing hydrogel, comprising:
a drug nanoparticle, comprising an active pharmaceutical ingredient encapsulated by a phospholipid-acrylate polymer; and
a porous hydrogel formed by crosslinking an acrylated biodegradable polymer with a pore-forming agent;
wherein the porous hydrogel is loaded with the drug nanoparticle.

2. The drug-releasing hydrogel of claim 1, wherein the drug-releasing hydrogel satisfies at least one of the following conditions:
(I) a mass ratio of the drug nanoparticle to the porous hydrogel is 1-30: 70-99;
(II) in the porous hydrogel, a mass of the pore-forming agent accounts for 20%-90% of a total weight of the porous hydrogel; or
(III) in the drug nanoparticle, a mass ratio of the phospholipid-acrylate polymer to the active pharmaceutical ingredient is 1:0.1-10.

3. The drug-releasing hydrogel of claim 1, wherein the phospholipid-acrylate polymer comprises a polymer formed by phospholipid, polyethylene glycol and acrylic acid.

4. The drug-releasing hydrogel of claim 3, wherein the phospholipid comprises at least one of 1,2-distearoyl-sn-glycero-3-phosphoethanolamine (DSPE), dipalmitoyl-phosphatidylethanolamine (DPPE), distearoylphosphocholine (DSPC), dimyristoyl-phosphatidylethanolamine (DMPE), 1,2-dioleoyl-sn-glycero-3-phosphoethanolamine (DOPE), 1,2-dioleoyl-sn-glycero-3-phosphocholine (DOPC), or poly(lactide-co-glycolide) (PLGA).

5. The drug-releasing hydrogel of claim 3, wherein a molecular weight of the polyethylene glycol is 1,000 Da to 25,000 Da.

6. The drug-releasing hydrogel of claim 1, wherein the acrylated biodegradable polymer comprises at least one of polyethylene glycol acrylate, polyethylene glycol methacrylate, polyethylene glycol diacrylate, disulfide-containing polyethylene glycol diacrylate, polyethylene glycol dimethacrylate, poly(2-hydroxyethyl methacrylate), poly(acrylic acid), polyacrylate, poly(methacrylic acid), or polymethacrylate.

7. The drug-releasing hydrogel of claim 6, wherein a number average molecular weight of the polyethylene glycol diacrylate is 500-8,000 Da.

8. The drug-releasing hydrogel of claim 1, wherein the pore-forming agent comprises at least one of polyethylene glycol, chitosan, agarose, dextran, hyaluronic acid, poly(methyl methacrylate), cellulose or a derivative thereof, gelatin or a derivative thereof, or acrylamide or a derivative thereof.

9. The drug-releasing hydrogel of claim 1, wherein the active pharmaceutical ingredient comprises at least one of an anti-inflammatory drug, an analgesic, or a local anesthetic.

10. The drug-releasing hydrogel of claim 1, wherein the active pharmaceutical ingredient further comprises a cytokine, a growth factor, or a wound healing agonist.

11. The drug-releasing hydrogel of claim 1, wherein the drug nanoparticle is spherical or quasi-spherical, and has a dimension of 50-200 nm.

12. The drug-releasing hydrogel of claim 1, wherein a zeta potential value of the drug nanoparticle is −35 mV to −5 mV.

13. The drug-releasing hydrogel of claim 1, wherein a diameter of pores in the porous hydrogel is 1-100 μm.

14. A method for preparing the drug-releasing hydrogel of claim 1, comprising the following steps:
adding a pore-forming agent, a drug nanoparticle solution and an initiator into an acrylated biodegradable polymer solution, and performing light curing to prepare the drug-releasing hydrogel.

15. The method for preparing the drug-releasing hydrogel of claim 14, wherein the drug nanoparticle solution is prepared by mixing an organic solution of a phospholipid-acrylate polymer with an organic solution of an active pharmaceutical ingredient, then dispersing a mixture of the two solutions into water, and sonicating the mixture to obtain the drug nanoparticle solution.

16. The method for preparing the drug-releasing hydrogel of claim 14, wherein the initiator comprises at least one of 2-hydroxy-2-methyl-1-phenyl-1-propanone, (2,4,6-trimethylbenzoyl)diphenylphosphine oxide, 1-hydroxycyclohexylphenyl ketone, 2,2-dimethoxy-phenylacetophenone, benzophenone, 2-isopropylthiothianthone, 2-methyl-1-(4-methylthiophenyl)-2-morpholino-1-propanone, 2-benzyl-2-dimethylamino-1-(4-morpholinophenyl)butanone, phenylbis(2,4,6-trimethylbenzoyl)phosphine oxide, a benzoyl formate mixture, 1,1′-(methylenebis(4,1-phenylene))bis(2-hydroxy-2-methylpropan-1-one), 2-hydroxy-4′-(2-hydroxyethoxy)-2-methylpropiophenone, lithium phenyl(2,4,6-trimethylbenzoyl) phosphinate, camphorquinone, ammonium persulfate, 2,2′-azobis(2-methylpropionamidine) dihydrochloride, riboflavin, or a combination of ammonium persulfate and N,N,N′,N′-tetramethylethylenediamine.

17. The method for preparing the drug-releasing hydrogel of claim 14, wherein the light curing is performed by crosslinking under ultraviolet light.

18. A drug for wound healing comprising the drug-releasing hydrogel of claim 1.

19. The drug for wound healing of claim 18, wherein the wound comprises at least one of a burn, a trauma, a wound left by surgical intervention, or a chronic wound.

20. A device for a wound, comprising the drug-releasing hydrogel of claim 1, and an optional substrate material.
